(19) United States
(12) Patent Application Publication
(10) Pub. No.: US 2005/0220264 A1
(43) Pub. Date: Oct. 6, 2005

(54) Method and device for medical image reconstruction

(75) Inventor: Joachim Homegger, Mohrendorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/089,251

(22) Filed: Mar. 24, 2005

(30) Foreign Application Priority Data
Mar. 31, 2004 (DE) 10 2004 016 586.6

(51) Int. Cl. G21K 1/12; H05G 1/60; A61B 6/00

(52) U.S. Cl. 378/8

(57) ABSTRACT

The present invention relates to an image reconstruction device (12) for an X-ray apparatus and a method for local 3D reconstruction of an object area of an examination object (7) from 2D image data of several 2D X-ray images of the examination object (7) registered in chronological order with different known projection geometries using the X-ray apparatus. With the method a location in the object area under consideration is selected from one of the 2D X-ray images. The positions of the selected location are determined in at least some of the 2D X-ray images and a spatial motion of the selected location between the registrations of the 2D X-ray images is calculated from the positions obtained, taking the known projection geometries into consideration. The calculated motion is then annulled by modifying the 2D image data in the 2D X-ray images and a 3D image dataset of at least the object area is reconstructed from the modified 2D image data. The method and the image reconstruction device enable a 3D image of a moving locally bounded object area to be reconstructed in a simple way without motion artifacts.

### CROSS REFERENCE TO RELATED APPLICATIONS

This application claims priority to the German application No. 10 2004 016 586.6, filed Mar. 31, 2004 which is incorporated by reference herein in its entirety.

### FIELD OF INVENTION

This invention relates to a method for local 3D reconstruction of an object area of an examination object from 2D image data of several 2D X-ray images of the examination object which were registered using an X-ray apparatus in chronological order with different known projection geometries. The invention also relates to an image reconstruction device for an X-ray apparatus with a reconstruction module which reconstructs a 3D image dataset of at least one object area from the 2D image data of these 2D X-ray images.

### BACKGROUND OF INVENTION

In imaging X-ray diagnostics, image acquisition using so-called C-arm devices plays an important role. With devices of this type, several 2D X-ray images of the examination object are registered under different projection geometries defined by the position of the C-arm and of the registration system disposed thereon. With a constant angular increment of the C-arm between successive registrations, methods of computer tomography can be used for three-dimensional reconstruction of the acquired area of the examination object subsequent to the image acquisition. With these reconstruction methods a 3D image dataset is obtained from the 2D image data of the image sequence of the 2D X-ray images, from which 3D image dataset any views of the area under examination can be generated and displayed on a monitor. However, the correct reconstruction of the acquired area requires that this area or objects contained therein do not move during the registration of the image sequence. A movement can result in disruptive image artifacts which prevent a diagnosis on the basis of the reconstructed area.

This problem occurs particularly with the generation of 3D image datasets from 2D X-ray images of a beating heart or of the associated vascular system for the assessment of vascular diseases. When analyzing coronary vascular diseases, stenoses and three-dimensional surveys thereof are very important, since for example a precise understanding of the 3D geometry enables a stent to be selected quickly and correctly for the treatment of the stenosis.

Various methods are currently used for surveying stenoses. Thus in many cases X-ray cardiology systems are supplied with quantification software, which enables the vessels to be surveyed in 2D X-ray images. However, surveying in this way only supplies two-dimensional information. Thus an exact three-dimensional survey is not possible. The doctor must himself transfer the results into three-dimensional space. The experience of the doctor providing the treatment thus becomes an important factor for a successful survey and for the soundly-based assessment of the survey results.

### SUMMARY OF INVENTION

In a further known technology, the 3D geometry of the vascular path is reconstructed from 2D X-ray images of the heart which were registered from different viewing directions or with different projection geometries. To this end recourse can be had to known methods from the area of research of computer vision, such as what are known as epipolar geometry or algorithms of stereo image processing, for example. However, these technologies provide only a basic structure of the vascular path. The vascular surface cannot be calculated using these methods. It can then merely be approximated, for example in that the vascular cross-section is modeled as an ellipse and the surface is parameterized using the parameters of the ellipse.

In a further known method a 3D reconstruction of the heart is obtained from X-ray images registered with a computer tomograph. These images permit, using a simultaneously acquired ECG (electrocardiogram), either a triggering of the image registration itself or a subsequent selection of image data which in each case corresponds to the same heart phase. Thus all projections can be collated to form a predefined heart phase and can be used for the reconstruction of the 3D image dataset. ECG triggering has until now been an important prerequisite for 3D image reconstruction of a beating heart.

U.S. Pat. No. 5,023,553 is concerned with motion compensation in NMR image registration. The method performs an image correlation of projection data contained in the image signals, in order then to correct the image data. The method requires the registration and correlation of projection data in addition to the actual image data, for which a modification of the registration method by an additional gradient field is required. Only by this modification is the projection data obtained which then has to be correlated in order to record the motion to be compensated for. This method cannot thus be applied to a generic method in the X-ray field.

E. H. W. Meijering et al., “Retrospective Motion Correction in Digital Subtraction Angiography: A Review”, IEEE Trans. on Medical Imaging, January 1999, pages 2-21, survey techniques of retrospective motion compensation in image data of digital subtraction angiography. Here, inter alia, the image content of successive 2D X-ray images is compared using a correlation technique, in order to record a motion of an object. On the basis of this recorded motion of an object, the images can then be corrected using geometric transformation. However, this method requires an identical registration geometry for all images in order to be able to derive the motion from the image correlation. In contrast the 2D X-ray images are registered from different projection directions in generic methods, so that such a procedure is not possible.

DE 102 06 190 A1 relates to a generic method for generation of a volume dataset. The object of this method is to simplify the generation of a volume dataset of an object in motion. This object is achieved in that a referencing base is arranged on the object in a defined way, and the position and orientation of this referencing base are acquired by a position acquisition system during the registration of the 2D X-ray images. Then for example the mapping of the object in the respective 2D projections on the basis of a deviation in the determined position and orientation of a reference position is corrected by displacing the mapping of the object in the 2D projections and thus the motion of the object is compensated for.

An object of the present invention is to specify a method for local reconstruction of an object area of an examination object from 2D image data of several 2D X-ray images of the examination object as well as an associated image reconstruction device for an X-ray apparatus, with which a 3D image dataset of an object area which moves during the registration of the 2D X-ray images can be generated in a simple way with sufficient image quality.

The object is achieved by the claims. Advantageous embodiments of the method and of the image reconstruction device form the subject of the dependent claims or can be taken from the following description and the embodiment.

In the present method for local reconstruction of an object area of an examination object from 2D image data of several 2D X-ray images of the examination object which were registered in chronological order with different known projection geometries with an X-ray apparatus, a location on the object area under consideration or a point of a stenosis is initially selected from one of the 2D X-ray images. The positions of the selected location are then determined in at least some of the 2D X-ray images. Preferably this determination takes place in all 2D X-ray images. A spatial motion of the selected location between the registrations of the 2D X-ray images is at least approximately calculated from the two-dimensional positions obtained in this way and the known projection geometries under which the corresponding 2D X-ray images were registered. This calculation thus produces a three-dimensional motion curve of the selected location during the registration of the image sequence. This motion is annulled by modifying the 2D image data in the 2D X-ray images (motion compensation). A 3D image dataset, which contains at least the object area under consideration, is then reconstructed from this modified 2D image data, taking the known projection geometries into consideration.

In the present method advantage is taken of the fact that sometimes only a locally narrowly bounded object area is of interest in the image registrations. Thus to survey a diseased vascular section it is not essential to reconstruct the coronaries in total. A local 3D reconstruction around a stenosis may suffice for the analysis. The basic concept of the proposed method is to track this special location under consideration in the individual X-ray images (object tracking), to calculate the spatial motion of this location from the image sequence and to adjust the 2D image data of the X-ray images from the three-dimensional to the two-dimensional of the object motion so that this location appears in the images without motion. Thus the location, for example the stenosis, which was in motion during the registration of the image sequence, is fixed, as it were frozen, in the images. In the 3D image dataset reconstructed from these modified images the location under consideration is then displayed without motion and can thus be identified very well while the surrounding object areas are reconstructed in motion. The only prerequisite for a successful reconstruction is that the object area to be reconstructed locally can be tracked across the registration sequence. This tracking can be done using image processing algorithms with minimal manual assistance or interaction with the user.

In an advantageous embodiment of the present method the positions of the selected location in the individual 2D X-ray images are hence tracked automatically using a pattern recognition method, so that no interaction with the user is required for this. In a further embodiment of the present method the object area under consideration and the location subsequently tracked can be automatically detected and determined by an image processing algorithm in the initial step. This is the case for example in the display of stenoses which can be discovered by an image processing algorithm by stipulating the parameters characterizing these in the image. For this, the image processing algorithm can for example segment the vascular path from the 2D X-ray image and identify local vascular constrictions by automatically surveying the diameter of the vessels along their path. A central point of the object area under consideration or a clearly defined point which can be particularly readily identified in the 2D X-ray images and thus can be traced can be used as a location to be tracked subsequently. Nor is it essential here for this to be a single image point. The location to be tracked can also consist of several image points. Should the first detection of the object area under consideration by the image processing algorithm not be possible, manual interaction with the user is of course also possible at any time. The user here selects, in one of the projections, i.e. the 2D X-ray images, a point to be tracked of the area under consideration. An image processing algorithm then tracks this point selected by the user across the image sequence.

After calculating the spatial motion of this point, for which generally all 2D X-ray images are used, the 2D image data of the 2D X-ray images is modified so that this point is no longer in motion in the image sequence of the modified 2D X-ray images. The motion of the point can here too of course only be calculated approximately. Further, depending on the time interval between the individual registrations in relation to the motion of the object, it is also possible to use not all X-ray images but only some of them, for example every second or third X-ray image, for object tracking. However, motion correction of course takes place in all 2D X-ray images used for the reconstruction.

The associated image reconstruction device for the X-ray apparatus for registering the 2D X-ray images comprises a reconstruction module which reconstructs a 3D image dataset of at least one object area from the 2D image data of several 2D X-ray images of an examination object registered in chronological order with different known projection geometries with the X-ray apparatus. The image reconstruction unit further comprises an object tracking module which determines positions of a definable location of an object area under consideration in at least some of the 2D X-ray images and calculates a spatial motion of the selected location between the registrations of the 2D X-ray images at least approximately from the positions, taking the known projection geometries into consideration. In a correction module connected to the object tracking module the calculated motion is annulled by modifying the 2D image data in the 2D X-ray images and the modified 2D image data for the reconstruction of the 3D image dataset from the modified 2D image data is made available to the reconstruction module. The image reconstruction unit can further additionally comprise a detection module which automatically detects and determines the object area under consideration and/or the definable location of the object area under consideration in the 2D X-ray images using an image processing algorithm in accordance with definable parameters.

The present method and the associated image reconstruction device permit local 3D reconstruction of a locally bounded object area on a cardiology system. A particular advantage of the method is the ease of execution, since ECG data is not required and no global motion model of the entire examination object has to be calculated. Reconstruction methods of standard C-arm computer tomography can be used directly for the reconstruction of the 3D image data without additional development expense.

### BRIEF DESCRIPTION OF THE DRAWINGS

The present method and the associated image reconstruction device are explained again below on the basis of an embodiment in conjunction with the drawings. These show:

FIG. 1 an example of a C-arm device with the present image reconstruction device; and

FIG. 2 a flowchart of a variant embodiment of the present method.

### DETAILED DESCRIPTION OF INVENTION

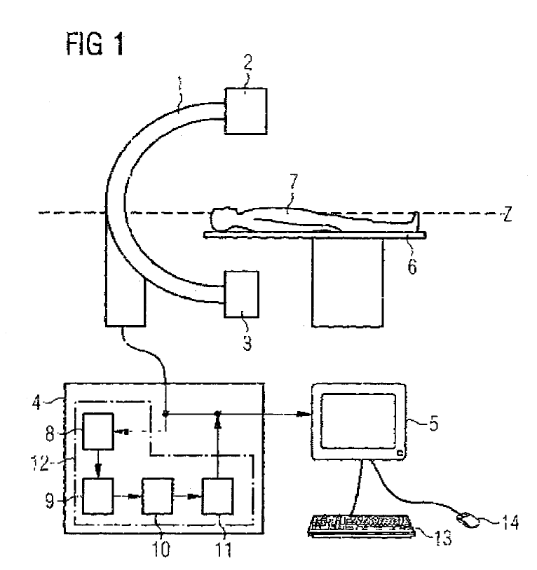

FIG. 1 shows in highly diagrammatic form a C-arm device for registering the 2D X-ray images. The C-arm device has a C-arm 1 that can rotate around the z-axis and to which are fixed an X-ray tube 2 and a detector 3 opposite the X-ray tube. The image data registered by the detector 3 at different rotational positions of the C-arm 1 is transmitted to the image processing unit 4, which is connected to a monitor 5 for displaying images of the registered or reconstructed images. This image processing unit 4 comprises, besides normal, not explicitly shown processing units, an image reconstruction device 12 with a detector module 8, an object tracking module 9, a correction module 10 and a reconstruction module 11, which are examined in greater detail below. The monitor is connected to a keyboard 13 and a graphic input device 14, via which a user can influence image display and image reconstruction.

This system also comprises the motor-adjustable patient table 6, on which the patient 7 to be examined lies during image registration. Rotating the C-arm 1 permits the device illustrated to register different projections of an examination area of the patient 7 as two-dimensional X-ray images.

In the present method the C-arm 1 traverses a circular path to generate the image sequence of the area under examination, in order to generate projection registrations in different projection directions. The angular increment of the rotation between consecutive registrations in each case is selected to be constant, in order to be able to apply standard methods of computer tomography for the reconstruction of the volume data. The projection geometry of this C-arm system must be calibrated before use, in order to know the precise projection geometries of each individual 2D X-ray image registered.

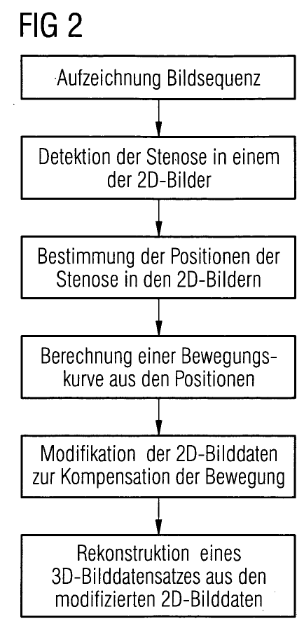

In the present example a stenosis in the patient's heart can be reconstructed locally in three dimensions, to enable a detailed survey of this stenosis. This is illustrated on the basis of the flowchart in FIG. 2 in conjunction with FIG. 1. The stenosis is detected with the help of an image processing algorithm in one of the registered 2D X-ray images and is determined for further tracking. This is done by the detection module 8 of the image reconstruction device 12 of the C-arm device. Then the stenosis or a point in the image display of this stenosis is localized in the object tracking module 9 with the image processing algorithm in the individual 2D X-ray images and its respective position is determined. From the positions determined a spatial motion curve of this point is calculated. For this, knowledge of the precise projection geometry of the respective 2D X-ray images is required, since only with a knowledge of these projection geometries stored in the image processing unit 4 can a spatial motion of the point be calculated.

Finally in the correction module 10 this calculated motion of the point is annulled in all 2D X-ray images. This is done as a function of the calculated direction of motion by displacing the image points of the respective 2D X-ray image and/or by changing the mapping scale of this image. This results in an image sequence of 2D X-ray images in which the determined point is not subject to any motion throughout the image sequence. This relates not only to the determined point, but also to the entire rigid area locally around the point and the stenosis. The greater the distance from this stenosis frozen in motion, the more noticeable the motion of the heart and the larger the reconstruction artifacts become in these remote areas during subsequent reconstruction.

The 2D image data modified in this way of the image sequence is passed to the reconstruction module 11, which reconstructs a 3D image dataset from this image data in a known way. The user can now generate any perspectives or sectional views from this 3D image dataset and display them on the monitor 5. In the illustration the determined stenosis appears without motion artifacts, whereas the more distant surroundings of this stenosis not under consideration have image artifacts caused by the motion.

1-12. (canceled)

13. A method of reconstructing a medical three-dimensional image of an object area of an examination object using two-dimensional image data, comprising:
recording a plurality of two-dimensional images of the examination object in chronological order using an x-ray device, the two-dimensional images having different projection geometries;
determining a movement of a sub-area included in the object area while recording the two-dimensional images;
compensating for the determined movement by modifying the two-dimensional image data included in the two-dimensional images; and
reconstructing a three-dimensional image of the object area using the modified two-dimensional image data, wherein the determining of the movement of the sub-area includes:
identifying the sub-area in one of the two-dimensional images,
determining a number of positions of the sub-area within a number of two-dimensional images other than the one two-dimensional image, and
calculating the movement of the sub-area using the determined number of positions and the projection geometries.

14. The method according to claim 13, wherein determining the number of positions includes applying a pattern recognition algorithm.

15. The method according to claim 13, wherein the object area and the sub-area are determined by an image processing algorithm based on a set of user-definable selection parameters.

16. The method according to claim 13, wherein the sub-area is a central location of the object area.

17. The method according to claims 13, wherein the sub-area is a striking location of the object area.

18. The method according to claim 13, wherein modifying the two-dimensional image data includes displacing image pixels of the two-dimensional images.

19. The method according to claims 13, wherein modifying the two-dimensional image data includes enlarging or reducing an image area of at least one of the two-dimensional images for adjusting the projection geometry of the two-dimensional image.

20. The method according to claim 13, wherein the x-ray device comprises a C-arm, and the two-dimensional images are recorded at different positions of the C-arm.

21. The method according to claim 13, wherein the object area is a heart, and the three-dimensional image visualizes a cardiac stenosis.

22. A device for medical image reconstruction for use with an X-ray device, comprising:
an image processing unit for reconstructing a three-dimensional image of an object area of an examination object using two-dimensional image data included in a plurality of two-dimensional images of the examination object, the two-dimensional images recorded by the X-ray device in chronological order and having different projection geometries;
an object tracking unit for determining a number of positions of a sub-area of the object area within a number of the two-dimensional images and for calculating a movement of the sub-area during the recording of the two-dimensional images using the determined positions and the projection geometries; and
a correction unit for compensating for the calculated movement by modifying the two-dimensional image data, the correction unit operatively connected to the image processing, wherein the image processing unit is configured to reconstruct the three-dimensional image using the modified two-dimensional image data.

23. The device according to claim 22, further comprising a detection unit for determining the object area or the sub-area based on an image processing algorithm and a set of user-definable selection parameters.

24. The device according to claim 23, wherein the object area is a stenosis of an organ and the detection module unit is configured to detect the stenosis.
